(19) United States
(12) Patent Application Publication
(10) Pub. No.: US 2022/0280188 A1
(43) Pub. Date: Sep. 8, 2022

(54) MEDICAL MICRODEVICE ANCHORING APPARATUS, INSTALLATION AND RETRIEVAL TOOLS, AND METHODS OF USING SAME

(71) Applicant: THE BRIGHAM AND WOMEN'S HOSPITAL, INC., Boston, MA (US)

(72) Inventors: Sharath Krishna Bhagavatula, Cambridge, MA (US); Kunj Bhaveshkumar Upadhayaya, Calgary (CA); Alex Andrew Lammers, Cambridgge, MA (US); Oliver Jonas, Weston, MA (US); Devon Michael Thompson, Ben Lomond, CA (US)

(21) Appl. No.: 17/637,182

(22) PCT Filed: Aug. 19, 2020
(86) PCT No.: PCT/US2020/046926

§ 371 Date: Feb. 22, 2022

Related U.S. Application Data
(60) Provisional application No. 62/889,239, filed on Aug. 20, 2019.

(30) Foreign Application Priority Data
Aug. 19, 2020 (US) PCT/US2020/046919

(51) Int. Cl. A61B 17/34; A61B 17/3205

(52) U.S. Cl. CPC A61B 17/3468; A61B 17/32053; A61B 2017/22049

(57) ABSTRACT

A microdevice includes a body having longitudinally spaced proximal and distal device ends and a guidewire extending proximally from the proximal device end. An anchoring apparatus includes an anchor having longitudinally spaced proximal and distal anchor ends. The anchoring apparatus is selectively expandable between a collapsed insertion condition and an expanded anchoring condition within the body tissue. A retrieval apparatus for selectively retrieving the medical microdevice and its associated anchoring apparatus from a target implantation site of a body tissue is described. The retrieval apparatus includes a stylet having proximal and distal stylet ends longitudinally separated by a stylet lumen configured to selectively accept the guidewire. A retrieval sheath has proximal and distal sheath ends longitudinally separated by a sheath lumen. The sheath lumen is configured to selectively and concurrently laterally surround the microdevice and the stylet therewithin for removal from the body tissue.

### RELATED APPLICATION

This application claims priority from U.S. Provisional Application No. 62/889,239, filed 20 Aug. 2019, the subject matter of which is incorporated herein by reference in its entirety. This application is also related to co-pending PCT Application No. PCT/US20/46919, filed concurrently herewith and entitled “MEDICAL MICRODEVICE ANCHORING APPARATUS, INSTALLATION AND RETRIEVAL TOOLS, AND METHODS OF USING SAME” (attorney docket no. PHC-028925 WO ORD 1); and to Bhagavatula S, Upadhyaya K, Miller B, Lammers A, Cima M, Silverman S, Jonas O, An interventional image-guided microdevice implantation and retrieval method for in-vivo drug response assessment, Medical Physics. 2019. 46(11):5134-5143; the contents of both of which are incorporated herein by reference in their entirety.

### GOVERNMENT SUPPORT

This invention was made with government support under one or more of grant numbers R21CA216796 and NIH R33CA223904. awarded by the National Institutes of Health. The government has certain rights in the invention.

### TECHNICAL FIELD

This disclosure relates to an apparatus and method for medical microdevice retrieval and, more particularly, to a method and apparatus for selectively retrieving a medical microdevice and associated anchoring apparatus from a target implantation site of a body tissue.

### BACKGROUND

In recent years, research has demonstrated that the progression of many diseases is governed by molecular and genetic factors which are patient-specific. For example, it is now understood that cancer may be driven by diverse genetic and epigenetic factors which are often patient-specific. As a result, disease progression and anti-cancer drug response are unique to every patient. In spite of this understanding, most clinical treatments still follow established standard-of-care guidelines and paradigms which fail to account for patient-specific factors.

Personalizing therapeutic treatments in view of patient-specific molecular and genetic factors offers the opportunity to improve therapeutic outcomes. In order to tailor treatments in a patient specific fashion, tools and methods of predicting and/or rapidly determining the response of a patient to particular drug regimens are needed.

Implantable microdevices can be used to locally deliver discrete microdose quantities of one or more active agents to tissues in a patient, and which can be easily removed along with a sample of adjacent patient tissue for later analysis and treatment planning.

### SUMMARY

In an aspect, a retrieval apparatus for selectively retrieving a medical microdevice and associated anchoring apparatus from a target implantation site of a body tissue is described. The microdevice includes a body having longitudinally spaced proximal and distal device ends and a guidewire extending proximally from the proximal device end. The anchoring apparatus includes an anchor having longitudinally spaced proximal and distal anchor ends. A selected one of the proximal and distal anchor ends is connected to a respective one of the distal and proximal device ends. The anchoring apparatus is selectively expandable between a collapsed insertion condition and an expanded anchoring condition within the body tissue. The retrieval apparatus includes a stylet having proximal and distal stylet ends longitudinally separated by a stylet lumen configured to selectively accept the guidewire. A retrieval sheath has proximal and distal sheath ends longitudinally separated by a sheath lumen. The sheath lumen is configured to selectively and concurrently laterally surround the microdevice and the stylet therewithin for removal from the body tissue.

In an aspect, a method for selectively retrieving a medical microdevice and associated anchoring apparatus from a target implantation site of a body tissue is described. The microdevice includes a body having longitudinally spaced proximal and distal device ends and a guidewire extending proximally from the proximal device end. The anchoring apparatus includes an anchor having longitudinally spaced proximal and distal anchor ends. A selected one of the proximal and distal anchor ends is connected to a respective one of the distal and proximal device ends. The anchoring apparatus is selectively expandable between a collapsed insertion condition and an expanded anchoring condition within the body tissue. A retrieval apparatus including a stylet having proximal and distal stylet ends longitudinally separated by a stylet lumen configured to selectively accept the guidewire is provided. The stylet lumen is configured not to accept the proximal device end thereinto. A retrieval sheath has proximal and distal sheath ends longitudinally separated by a sheath lumen. A proximal end of the guidewire is inserted into the stylet lumen. The stylet lumen is guided toward the target implantation site over the guidewire by passing the stylet lumen distally along the guidewire. The proximal device end is contacted with the distal stylet end. The distal stylet end is maintained in contact with the proximal device end while accepting the stylet and enclosed guidewire into the sheath lumen. The sheath lumen is passed distally along the stylet and into a position within the body tissue such that the microdevice is at least partially enclosed in the sheath lumen within the body tissue. The microdevice is laterally surrounded within the sheath lumen. Distal movement of the sheath lumen past the anchoring apparatus is prevented by at least one of contacting the anchoring apparatus with the distal sheath end and accepting at least a portion of the anchoring apparatus into the sheath lumen. The microdevice and stylet are maintained in the sheath lumen. The sheath lumen, with the microdevice and stylet carried therein, is retracted proximally from the target implantation site. The. The microdevice and stylet are removed within the sheath lumen from the body tissue.

In an aspect, a retrieval apparatus for selectively retrieving a medical microdevice and associated anchoring apparatus from a target implantation site of a body tissue is described. The microdevice includes a body having longitudinally spaced proximal and distal device ends and a guidewire extending proximally from the proximal device end. The anchoring apparatus includes an anchor having longitudinally spaced proximal and distal anchor ends. A selected one of the proximal and distal anchor ends is connected to a respective one of the distal and proximal device ends. The anchoring apparatus is selectively expandable between a collapsed insertion condition and an expanded anchoring condition within the body tissue. The retrieval apparatus includes a stylet having proximal and distal stylet ends longitudinally separated by a stylet lumen configured to selectively accept the guidewire. The stylet lumen is configured not to accept the proximal device end thereinto. A retrieval sheath has proximal and distal sheath ends longitudinally separated by a sheath lumen. The distal sheath end is configured for selective contact with the anchoring apparatus. The distal sheath end includes a concentric cutting edge for coring the body tissue. The sheath lumen is configured to selectively and concurrently laterally surround the microdevice, the stylet, and a biopsy sample portion of the body tissue immediately adjacent the microdevice therewithin for removal from the body tissue. A housing is operatively connected to the proximal stylet end and the proximal sheath end. The housing is configured for selective manipulation by a user to independently longitudinally move the stylet and the sheath.

### BRIEF DESCRIPTION OF THE DRAWINGS

For a better understanding, reference may be made to the accompanying drawings, in which:

FIG. 1 is a schematic side view of an apparatus according to an aspect of the invention;

FIG. 2 is a schematic side view of the apparatus of FIG. 1 in a first example configuration;

FIGS. 3A-3B schematically depict an example sequence of operation of the apparatus of FIG. 2;

FIGS. 4A-4B are schematic side views of the apparatus of FIG. 1 in additional example configurations;

FIG. 5A schematically depicts the apparatus of FIG. 1 in an example delivery configuration;

FIGS. 5B-5D are schematic side views of components of a delivery system for the apparatus of FIG. 1;

FIG. 5E schematically depicts an assembled delivery system including the apparatus of FIG. 1;

FIGS. 5F-51 schematically depict an example sequence of delivery of the apparatus of FIG. 1;

FIGS. 6A-6C schematically depict an example sequence of delivery of the apparatus of FIG. 1;

FIG. 7 is a schematic perspective top view of a tool for use with the apparatus of FIG. 1;

FIGS. 8A-8C are schematic side views of components of the tool of FIG. 7;

FIGS. 9A-9C are perspective top views schematically depicting use of the tool of FIG. 7 with the apparatus of FIG. 1;

FIGS. 10A-10C schematically depict an example sequence of retrieval of the apparatus of FIG. 1; and

FIGS. 11A-11E are schematic partial side views depicting use of the tool of FIG. 7 with the apparatus of FIG. 1.

### DESCRIPTION OF ASPECTS OF THE DISCLOSURE

Unless defined otherwise, all technical and scientific terms used herein have the same meaning as is commonly understood by one of ordinary skill in the art to which the present disclosure pertains.

As used herein, the term “patient” can be used interchangeably with the term “subject” and refer to any living organism including, but not limited to, human beings, pigs, rats, mice, dogs, goats, sheep, horses, monkeys, apes, rabbits, cattle, farm animals, livestock, reptiles, fish, etc.

As used herein, the singular forms “a”, “an”, and “the” can include the plural forms as well, unless the context clearly indicates otherwise. It will be further understood that the terms “comprises” and/or “comprising”, as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

It will be understood that when an element is referred to as being “on”, “attached” to, “connected” to, “coupled” with, “contacting”, “adjacent”, etc., another element, it can be directly on, attached to, connected to, coupled with, contacting, or adjacent the other element, or intervening elements may also be present. In contrast, when an element is referred to as being, for example, “directly on”, “directly attached” to, “directly connected” to, “directly coupled” with, “directly contacting”, or “directly adjacent” another element, there are no intervening elements present. It will also be appreciated by those of ordinary skill in the art that references to a structure or feature that is disposed “directly adjacent” another feature may have portions that overlap or underlie the adjacent feature, whereas a structure or feature that is disposed “adjacent” another feature might not have portions that overlap or underlie the adjacent feature.

Spatially relative terms, such as “under”, “below”, “lower”, “over”, “upper”, “proximal”, “distal”, and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as “under” or “beneath” other elements or features would then be oriented “over” the other elements or features.

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that, although the terms “first”, “second”, etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

The invention comprises, consists of, or consists essentially of the following features, in any combination.

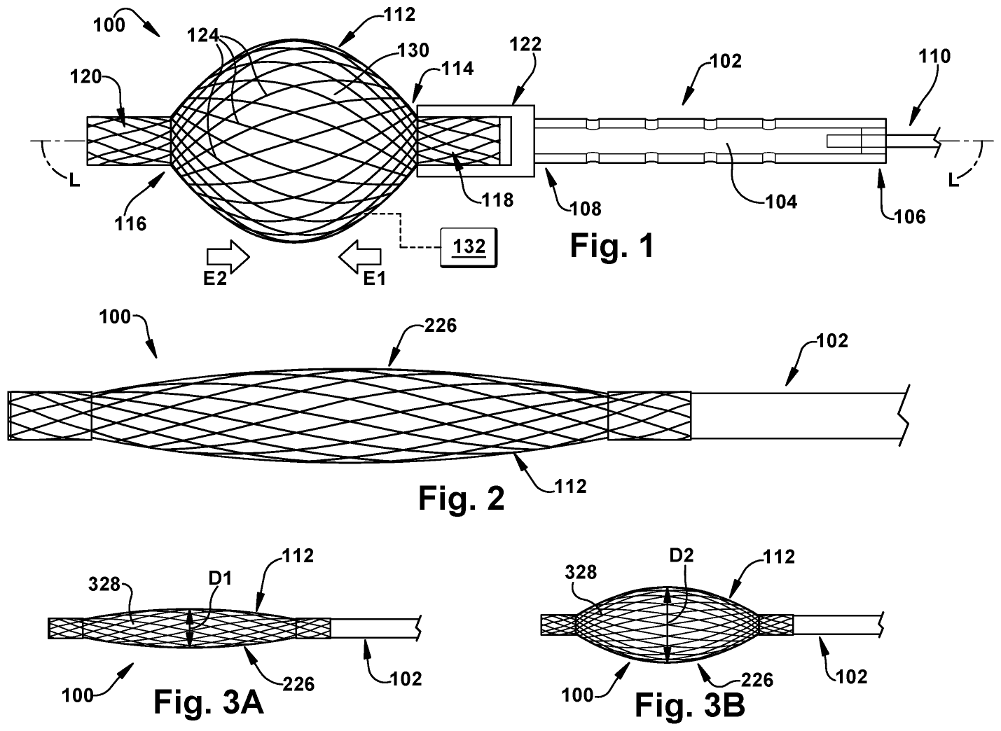

FIG. 1 depicts an anchoring apparatus 100 for a medical microdevice 102. The medical microdevice 102 may be similar, for example, to those disclosed in U.S. Pat. No. 10,183,155, issued 22 Jan. 2019 to Robert I. Tepper et al., the entirety of which is incorporated herein by reference. The medical microdevice 102 includes a body 104 having longitudinally spaced proximal and distal device ends 106 and 108, respectively, and a guidewire 110 extending proximally from the proximal device end 106. The term “guidewire” is used herein to include an elongate structure of any desired material and flexibility, including, but not limited to, a wire, suture, tube, any other desired component, or any combination thereof. The “longitudinal” direction, as referenced herein, is substantially parallel to axis L in FIG. 1, which is substantially in the horizontal direction in the orientation of that Figure.

The anchoring apparatus 100 includes a selectively expandable anchor 112 having longitudinally spaced proximal and distal anchor ends 114 and 116, respectively. At least one of the proximal and distal anchor ends 114 and 116 is connected, and may be directly connected, to a selected one of the proximal device end 106 and the distal device end 108. (For ease of depiction, the description herein presumes that the proximal anchor end 114 is connected to the distal device end 108.) Accordingly, the expandable anchor 112 may extend coaxially distally (or proximally) from the medical microdevice 102. The term “coaxial” is used herein to indicate that the expandable anchor 112 and medical microdevice 102 may share a common center about a longitudinal axis L. Alternatively, the expandable anchor 112 could be at least partially offset from the axis L of the medical microdevice 102. It is also contemplated, as previously alluded to, that the expandable anchor 112 and/or an additional expandable anchor (not shown) may extend—coaxially or at least partially offset—proximally from the medical microdevice 102.

As depicted in FIG. 1, the expandable anchor 112 may include proximal and distal anchor hubs 118 and 120, respectively. The proximal anchor hub 118 may be directly attached to the distal device end 108 or, as shown, the proximal anchor hub 118 could be received in socket 122 connected to the distal device end 108. One of ordinary skill in the art is readily able to provide a suitable mechanical connection between the expandable anchor 112 and the medical microdevice 102.

As will be depicted in more detail below, the anchor 112 selectively self-expands from a collapsed insertion condition to an expanded anchoring condition after insertion into a body tissue to resist post-implantation motion of the microdevice 102, which could include, but is not limited to, at least one of distal motion, proximal motion, rotation about the longitudinal axis, and lateral motion. When present, the proximal and distal anchor hubs 118 and 120 may move longitudinally relatively toward one another (i.e., in directions E1 and E2, respectively) during expansion of the expandable anchor 112. That is, the expandable anchor 112 can enlarge or expand in the radial direction while contracting in the longitudinal direction during overall “expansion”. The reverse is also contemplated for some configurations of the anchor 112—that the proximal and distal anchor hubs 118 and 120 move longitudinally relatively away from one another during expansion of the expandable anchor 112. The “radial” direction, as used herein, is substantially perpendicular to axis L, and extends into and out of the plane of the page, in the orientation of FIG. 1. Self-expansion could be provided in any desired manner, such as, but not limited to, the use of a shape-memory material and/or release of stored energy from a mechanically constrained structure.

Again, as shown in FIG. 1, the expandable anchor 112 may include a plurality of struts 124, with each strut being formed from a self-expanding material such as, but not limited to, NiTinol. Each strut 124 extends substantially longitudinally in the collapsed insertion condition, and is configured for selective self-expansion from the collapsed insertion condition to the expanded anchoring condition within the body tissue. One example commercially available device which could be used as at least a portion of a strut-type expandable anchor 112 is the Tumark Vision, available from SOMATEX® Medical Technologies GmbH of Berlin, Germany.

The expandable anchor 112 may also or instead include a mesh tube 226 at least partially formed from a self-expanding material. An example of a mesh tube 226 which may be suitable for certain use environments is commercially available as the SecurMark® Biopsy Site Marker from Hologic, Inc. of Marlborough, Mass. The mesh tube 226, when present, may extend substantially longitudinally in the collapsed insertion condition, and is configured for selective self-expansion from the collapsed insertion condition to the expanded anchoring condition within the body tissue. An example expandable anchor 112 comprising a mesh tube 226 is shown in FIG. 2.

FIGS. 3A-3B schematically depict an example situation wherein the expandable anchor 112 includes a mesh tube 226 carrying an expandable hydrogel 328 or other expandable material therein. In this configuration of the expandable anchor 112, the mesh tube 226 extends substantially longitudinally in the collapsed insertion condition, and is at least partially configured for selective expansion from the collapsed insertion condition to the expanded anchoring condition within the body tissue due to externally-directed force upon the mesh tube 226 from post-implantation expansion of the hydrogel 328 or other expandable material. That is, and as depicted by diametric arrows D1 and D2 in FIGS. 3A and 3B, respectively, the hydrogel 328 or other expandable material absorbs fluid within the body tissue and “plumps up” to increase the volume carried within the mesh tube 226. It is contemplated that the mesh tube 226, when carrying an associated hydrogel 328, could be made from either a simple non-expanding material or at least partially formed from a self-expanding material.

With reference now back to FIG. 1, the expandable anchor 112 may include an expandable balloon 130 (whether or not one or more struts 124 are present) having a fluid reservoir (shown schematically at 132) associated therewith. When present, the expandable balloon 130 may be configured for expansion from the collapsed insertion condition to the expanded anchoring condition within the body tissue due to transfer of fluid from the fluid reservoir 132 to the balloon 130.

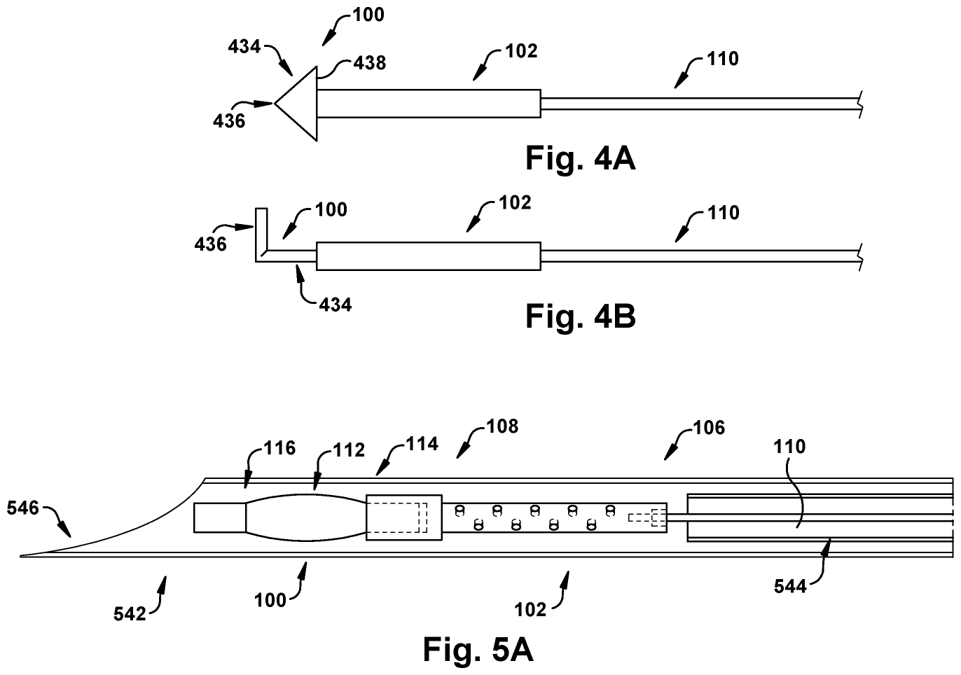

The expandable anchor 112 may, in some embodiments, be a first anchor, and the anchoring apparatus 100 may also include a second mechanical anchor 434 configured to additionally resist post-implantation motion of the medical microdevice 102. Examples of such second mechanical anchors 434 are shown in FIGS. 4A-4B, with the expandable anchor 112 omitted from those views, for clarity. For example, and as shown in FIG. 4A, the second mechanical anchor 434 has a conical aspect with an apex 436 located at a distalmost portion thereof and a round base 438 connected to the distal device end 108. As another example, and is shown in FIG. 4B, the second mechanical anchor 434 may include one or more angled wires 440 made at least partially of a self-expanding material which moves inside the body tissue from a straight insertion configuration (not shown) to an bent anchoring configuration. It is also contemplated that one or more of the “second” mechanical anchors 434—whether or not self-expanding—could be provided as an anchoring apparatus 100 without an accompanying (first) expandable anchor 112.

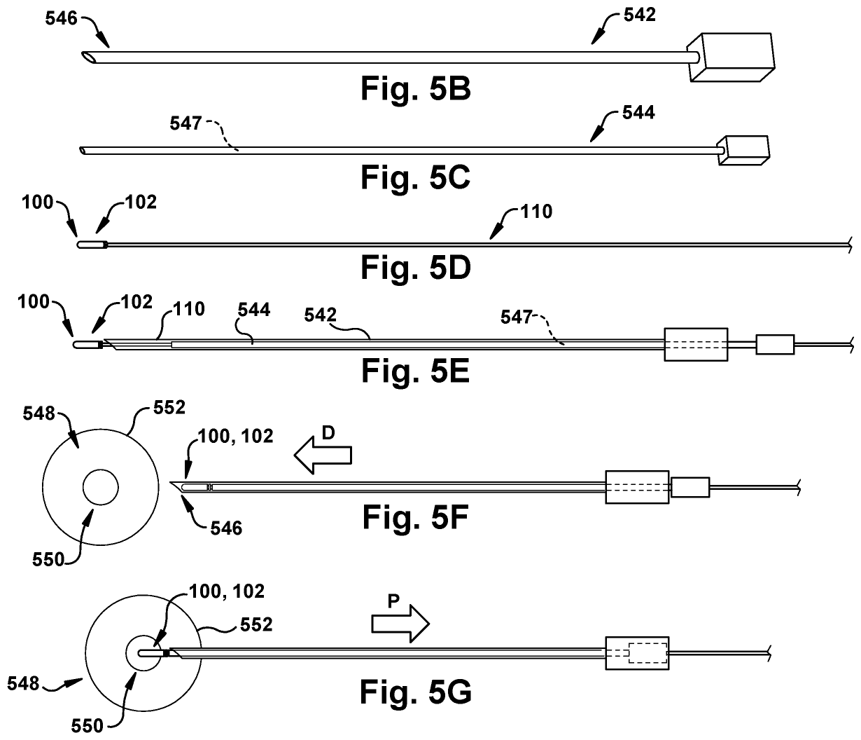

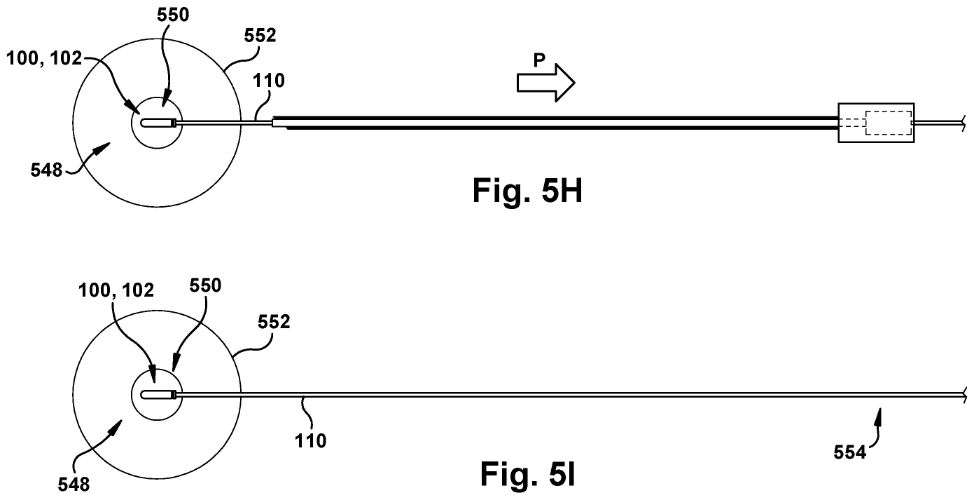

FIGS. 5A-51 schematically depict examples of various components of a delivery system for the medical microdevice 102 and attached anchoring apparatus 100. For example, FIG. 5A depicts the medical microdevice 102 and attached anchoring apparatus 100 in a collapsed insertion condition within an insertion needle 542. Optionally, and as shown, an inner stylet 544 could be provided to help maintain the medical microdevice 102 and attached anchoring apparatus 100 in a desired longitudinal position within the insertion needle 542, in any desired manner. The insertion needle 542 may include a tapered and/or sharpened distal needle end 546 to assist with piercing, cutting, and/or atraumatically shifting the body tissue to allow ingress of the insertion needle 542.

FIG. 5B schematically depicts an example insertion needle 542 which could be used with the delivery system. FIG. 5C schematically depicts an example inner stylet 544 which could be used with the delivery system. In some use environments, a known clinical insertion needle system could be used as a starting point to provide one or both of the insertion needle 542 and inner stylet 544. One suitable clinical insertion needle system is a Chiba biopsy needle, available from Cook Medical of Bloomington, Ind., However, the inner stylet 544 shown herein differs from that of the known insertion needle system, in that the inner stylet 544 of the delivery system shown in the Figures includes a hollow stylet lumen 547 extending longitudinally at least partially therethrough to selectively accept the guide wire 110 of the medical microdevice 102.

FIG. 5D schematically depicts a medical microdevice 102 and attached anchoring apparatus 100 and guide wire 110. In FIG. 5E, the component of FIG. 5B-5D are shown as assembled into a “loaded” delivery system. That is, the inner stylet 544 is inserted into the insertion needle 542, and the guide wire 110 of the medical microdevice 102 is accepted into the stylet lumen 547.

The insertion needle 542 and/or inner stylet 544 can be manipulated to encompass the medical microdevice 102 and anchoring apparatus 100 within the insertion needle 542, as shown in FIG. 5F, for insertion into a body tissue 548 toward a target implantation site 550. This may be accomplished by advancing the insertion needle 542 of the “loaded” delivery system through the skin surface 552, as shown by distal arrow D in FIG. 5F, and puncturing the body tissue 548 with the distal needle and 546.

In FIG. 5G, the medical microdevice 102 and anchoring apparatus 100 are shown in the process of being released from the delivery system at the target implantation site 550. This may be accomplished via at least one of proximal motion (signified by proximal arrow P in FIG. 5G) of the insertion needle 542, distal motion of the inner stylet 544 to “push” the medical microdevice 102 and anchoring apparatus 100 distally from the delivery system, or in any other desired manner that facilitates relative longitudinal motion between the insertion needle 542 and the medical microdevice 102. With reference now to FIG. 5H, the delivery system (comprising the insertion needle 542 and the inner stylet 544) are being concurrently withdrawn proximally from the body tissue 548, through the skin surface 552. Finally, in FIG. 5I, the medical microdevice 102 and attached anchoring apparatus 100 are maintained at the target implantation site 550 within the patient's body, while the insertion needle 542 and inner stylet 544 have been completely removed.

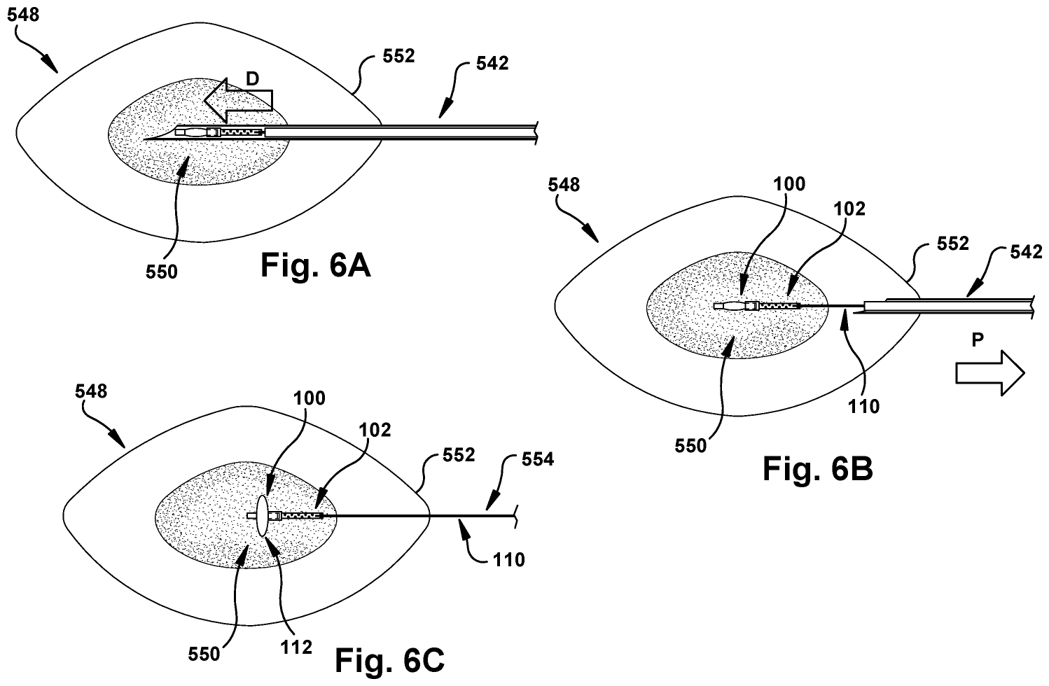

FIGS. 6A-6C also schematically depict an example method of selectively maintaining a medical microdevice 102 in a predetermined position within a body tissue 548, including description of use of the anchoring apparatus 100. The medical microdevice 102 has an associated anchoring apparatus 100, which may be substantially similar to those previously described. The proximal anchor end 114 is connected to the proximal device end 106 or the distal device end 108.

The microdevice 102 and attached anchoring apparatus 100 are at least partially enclosed, in a collapsed insertion condition, within an insertion needle 542. This is a similar arrangement to that shown in FIG. 5. As shown in FIG. 6A, the insertion needle 542, with the microdevice 102 and attached anchoring apparatus 100 at least partially enclosed therein, is advanced longitudinally distally (as shown by arrow D) into the body tissue 548 toward a target implantation site 550. This advancement can include puncturing the skin surface 552 overlying the body tissue 548.

At the target implantation site 550, longitudinal position of the insertion needle 542, with the microdevice 102 and attached anchoring apparatus 100 at least partially enclosed therein, is maintained for a desired length of time. It is contemplated that, for most use environments of the anchoring apparatus 100, this maintenance of position of the insertion needle 542 at the target implantation site 550 will be relatively brief and may be, for example, correlated with a length of time appropriate to confirm that the insertion needle 542 has reached the implantation site 550 via measurement, imaging, or any other desired confirmation scheme.

The insertion needle 542 can then be slightly retracted, in the proximal direction (arrow P in FIG. 6B), with or without an accompanying retraction of the inner stylet 544, to allow the anchoring apparatus 100 to self-expand from the collapsed insertion condition to an expanded anchoring condition at the target implantation site 550. This expansion may occur in any desirable manner, such as, but not limited to, actuation of a shape-memory material (e.g., a NiTinol strut 124, mesh tube 226, or any other structure) due to temperature of the body tissue 548, release of a previously mechanically constrained anchor 112 toward a pre-biased expanded condition, inflation of a balloon-type anchor 112, or any other desired expansion scheme. Examples of some suitable expansion structures and mechanisms were previously discussed with reference to FIGS. 1-4B, or one of ordinary skill in the art could readily provide a desired mechanism for expanding the anchor 112, over any desired indwelling time (including, but not limited to, immediately upon retraction of the insertion needle 542 and/or after passage of sufficient time for a fluid to migrate from the body tissue 548 into a hydrogel 328) for a particular use environment. In the particular example shown in FIGS. 6A-6C, the anchor 112 is depicted as indwelling within the body tissue 548 while the insertion needle 542 is retracted, before the anchor 112 reaches a fully expanded anchoring condition in the view of FIG. 6C.

However, it is contemplated that at least some slight expansion of the anchoring apparatus 100 toward the expanded anchoring condition may occur to help maintain the medical microdevice 102 at the target implantation site 550, in addition to, or instead of, relative motion between the inner stylet 544 and the insertion needle 542 “pushing” the medical microdevice 102 forward out of the insertion needle 542 in any event, the insertion needle 542 may be retracted longitudinally proximally, including entirely out of engagement with the body tissue 548, after the anchoring apparatus 100 is at least partially in the expanded anchoring condition, to release the microdevice 102 at the target implantation site 550. At that point, the anchoring apparatus 100 is at least partially in the expanded anchoring condition, and is operative to resist post-implantation motion or migration of the microdevice 102 away from the target implantation site 550. This is the situation shown in FIG. 6C.

Again, with reference to FIG. 6C, when the microdevice 102 is at the target implantation site 550, the guidewire 110 may at least partially protrude proximately from the body tissue 548 toward the skin surface 552 with a proximal guidewire end 554 located in ambient space adjacent the skin surface 552. Stated differently, the guidewire 110 may at least partially remain outside the patient's body for any desired length of time, and then may be used to provide a positional indication to a user of the location of the microdevice 102 within the patient's body tissue 548, as will be discussed in more detail below. In most use environments, a gauze pad, medical tape, an adhesive bandage, a catheter securement dressing, an anchoring point for a suture-type guidewire 110, and/or any other desired covering may be provided to protect the protruding guidewire 110 from contact with surfaces in the ambient space, prevent abrasion of the skin surface 552 by the protruding guidewire 110, visually obscure the penetration site to avoid patient discomfort, prevent ingress of bacteria and other microorganisms into the patient's body, or for any other desired reason.

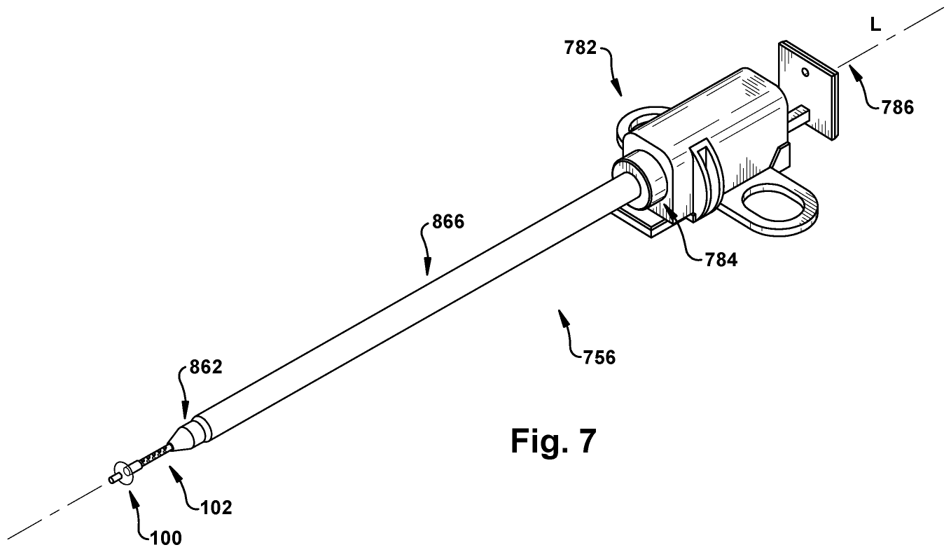

FIGS. 7-11E depict the components, structures, and example sequences of use of a retrieval apparatus 756 which can be used with the anchoring apparatus 100 and medical microdevice 102 of FIGS. 1-6C. The retrieval apparatus 756 is provided for selectively retrieving a medical microdevice 102, adjacent body tissue 548, and/or associated anchoring apparatus 100 from a target implantation site 550 of a body tissue 548.

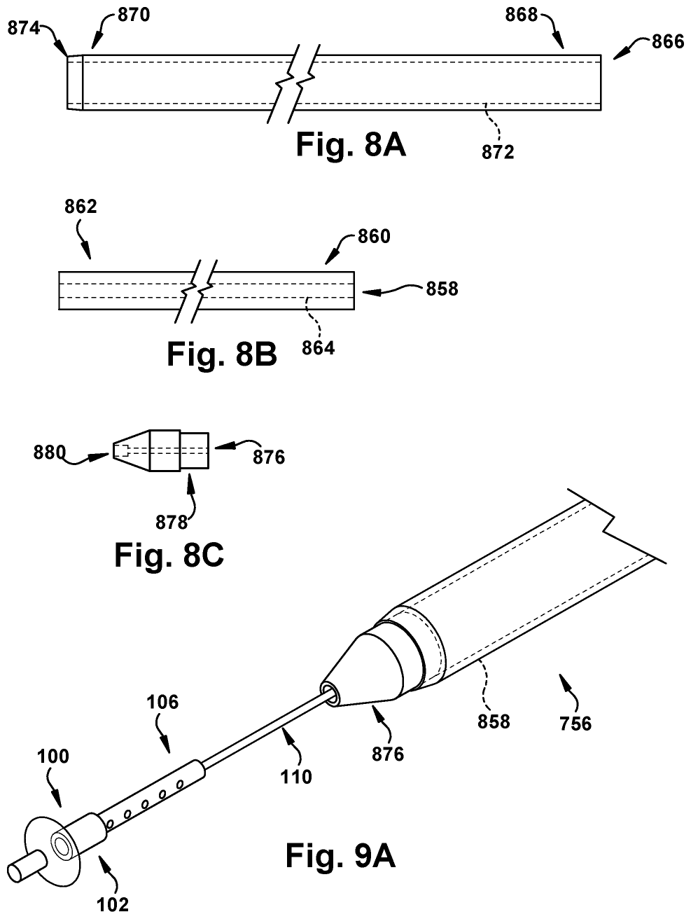

The retrieval apparatus 756 includes several components which are shown in FIGS. 8A-8C. A stylet 858 has proximal and distal stylet ends 860 and 862, respectively, longitudinally separated by a stylet lumen 864 configured to selectively accept the guidewire 110. The stylet lumen 864 may be smaller than the proximal device end 106, or otherwise configured the stylet lumen 864 does not accept the proximal device end 106 thereinto, in certain use environments. In other use environments, at least a portion of the medical microdevice may be selectively accepted into the stylet lumen 864, as desired.

A retrieval sheath 866 has proximal and distal sheath ends 868 and 870, respectively, longitudinally separated by a sheath lumen 872. The sheath lumen 872 is configured to selectively and concurrently laterally surround the microdevice 102 and the stylet 858 therewithin for removal from the body tissue 548. That is, the microdevice 102 and stylet may be held within the sheath lumen 872 during retrieval/removal of the microdevice 102 from the body tissue 548. As shown in FIG. 8A, the distal sheath end 870 may include a concentric cutting edge 874, which may be provided for coring or coring through the body tissue 548 under longitudinally directed force during insertion of the retrieval apparatus 756 into the body tissue 548.

As depicted in FIG. 8C, at least one of the distal stylet end 862 and the distal sheath end 870 may include a tapered nosecone 876 for atraumatically engaging the body tissue 548 during insertion of the retrieval apparatus 756 into the body tissue 548. It is contemplated that the concentric cutting edge 874 could be configured, in some use environments, to taper backward from a nosecone 876 provided to the stylet 858, during insertion of the retrieval apparatus 756, and thus ease insertion of the sheath 866 into the body tissue 548, even if the concentric cutting edge 874 does not actually sever the body tissue 548 during insertion. The nosecone 876 depicted is of a “plug” type, having a stub end 878 which is adhered to, press-fit, or otherwise placed into engagement with, e.g., the stylet lumen 864, but may be configured as desired for a particular use environment by one of ordinary skill in the art. When a nosecone 876 is present, the stylet lumen 864 could be larger than a diameter of the proximal device end 106, though it is contemplated that—in some use environments—the nosecone 876 will have an inner nosecone lumen which is too small to accept the medical microdevice 102 at least partially thereinto.

Also as shown in FIG. 8C, the distal stylet end 862 (which, as previously noted, may include a nosecone 876 as desired) may include a device notch 880. When present, the device notch 880 may selectively longitudinally accept a portion of the proximal device end 106 in a predetermined orientation, as will be discussed below.

With reference back to FIG. 7, the retrieval apparatus 756 may include a housing 782 which is operatively connected to the proximal stylet end 860 and/or the proximal sheath end 868. The housing 782, when present, may be configured for selective manipulation by a user to independently longitudinally move the stylet 858 and the sheath 866, as desired.

The housing 782 may include a clamp (shown schematically at 784) for selectively grasping at least a portion of the guidewire 110, such as the proximal guidewire end 554, as will be discussed below, to maintain the device body 104 in substantially constant relative longitudinal spacing from the housing 782 during the retrieval process. The clamp 784, when present, may be of any desired type, including, but not limited to, a turnscrew, a frictionally engaged grasper, pinching jaws, a tightenable collar, a spring-biased jaw, any other desirable scheme for holding the proximal guidewire end 554, or any combination thereof.

As shown in FIG. 7, the stylet 858 and sheath 866 may be mutually coaxially concentric when assembled into a ready-to-use retrieval apparatus 756. That is, the stylet 858 may fit into the sheath lumen 872 such that the two structures share a common longitudinal axis L. It is contemplated, though, that for most use environments, the stylet 858 and sheath 866 will be configured such that they can easily slide longitudinally relative to each other, optionally with slight frictional force therebetween which requires a user to intentionally move one relative to the other to effect such sliding.

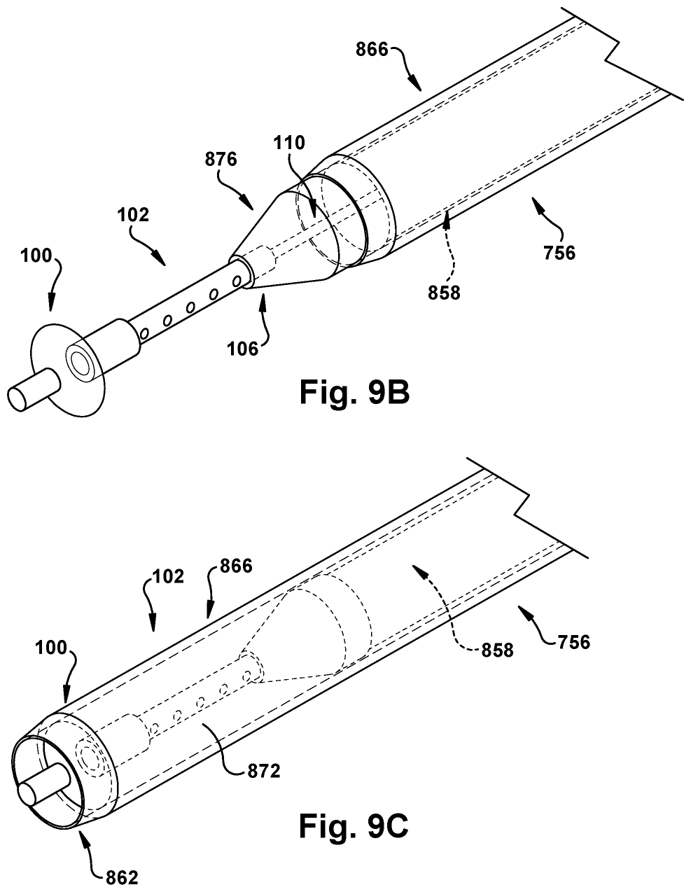

FIGS. 9A-9C schematically depicts an example sequence of operation of the retrieval apparatus 756, and how the retrieval apparatus 756 interacts with the medical microdevice 102 and the anchoring apparatus 100. As shown in FIG. 9A, the stylet 858 (here, with a leading nosecone 876) accepts a proximal guidewire and 76 and is guided along the guidewire 110, as along a rail, until the distalmost portion of the stylet 858 assembly (here, a distal end of the nosecone 876) reaches the proximal device end 106. Optionally, and as shown in FIG. 9B, a device notch 880 or other portion of the stylet 858 assembly (here, a portion of the nosecone 876) accepts the proximal device end 106 therein.

Then, as shown in FIG. 9B, the sheath 866 is longitudinally slid distally past the distalmost extent of the stylet 858 assembly, to at least partially enclose the medical microdevice 102. As shown in the configuration of FIG. 9C, the anchoring apparatus 100 (shown in the expanded condition in FIGS. 9A-9C) may be at least partially surrounded within the sheath lumen 872 concurrently with the sheath lumen 872 laterally surrounding the microdevice 102 and stylet 858. Then, once the retrieval apparatus 756 is carrying the anchoring apparatus 100 and medical microdevice 102 within its sheath lumen 872—that is, in the arrangement shown schematically in FIG. 9C—the retrieval apparatus 756 can be withdrawn from the patient's body to remove the medical microdevice 102 therefrom.

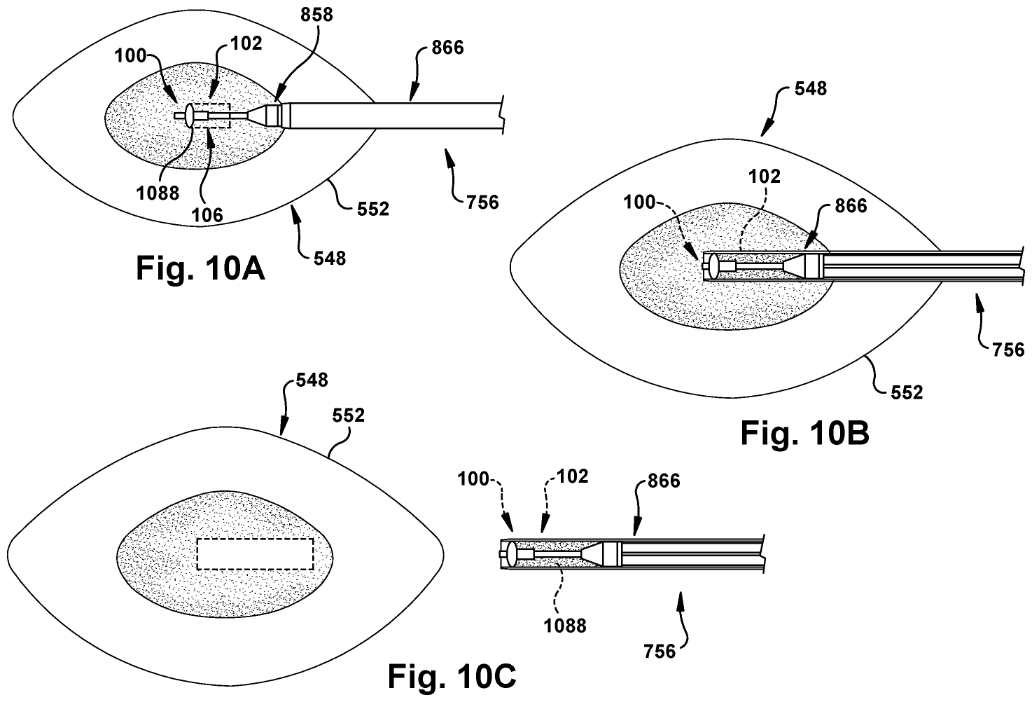

Another example of sequence of operation of the retrieval apparatus 756 is shown schematically in FIGS. 10A-10C. In these Figures, the microdevice 102 has been implanted in a body tissue 548 with the guidewire 110 protruding proximally through a skin surface 552 from the body tissue 548 into ambient space adjacent the skin surface 552. The microdevice 102 is maintained in the implanted position via resistance of the anchoring apparatus 100, in the expanded anchoring condition, to longitudinal motion of the microdevice 102, as previously described. As shown in FIG. 10A, the stylet 858 has been engaged with the guidewire 110 in the ambient space. The stylet 858 is then subject to guidance by the guidewire 110 toward the body tissue 548 immediately adjacent the proximal device end 106. In this manner, the retrieval apparatus 756 can be accurately guided to a previously implanted medical microdevice 102 buried beneath the skin surface 552. That is, the retrieval apparatus 756 is simply guided, as along a rail, through any tortuous anatomy which might happen to be present and straight to the medical microdevice 102. Optionally, imaging can be performed to confirm positioning and location of the microdevice 102, retrieval apparatus 756 (or portions thereof), or any other component of the system before retrieval is commenced. To this end, the anchoring apparatus 100 and/or any other portion of the system may be at least partially radiopaque, to assist with easy image interpretation.

In the example sequence of operation of FIGS. 10A-10C, the retrieval apparatus 756 has been configured to retrieve a biopsy sample 1088 portion of the body tissue 548 immediately adjacent the microdevice 102, for testing, isolation, or any other desired purpose. In order to obtain such a biopsy sample 1088, the retrieval apparatus 756 is configured to at least partially laterally surround the biopsy sample 1088 within the sheath lumen 872 concurrently with the sheath lumen 872 laterally surrounding the microdevice 102 and stylet 858.

This biopsy sample 1088 portion of the procedure, when present, could be aided by the concentric cutting edge 874 of the distal sheath end 870 cutting or coring through the body tissue 548 when the sheath 866 is advanced distally to surround the microdevice 102. In some use environments, the retrieval apparatus 756 could be configured with the sheath lumen 872 being smaller than the anchoring apparatus 100, when the anchoring apparatus 100 is in the expanded anchoring condition. As a result, the distal sheath end 870, with any present concentric cutting edge 874, can be brought into contact with a proximally-facing surface of the expanded anchoring apparatus 100, to help define a distalmost extent of the biopsy sample 1088.

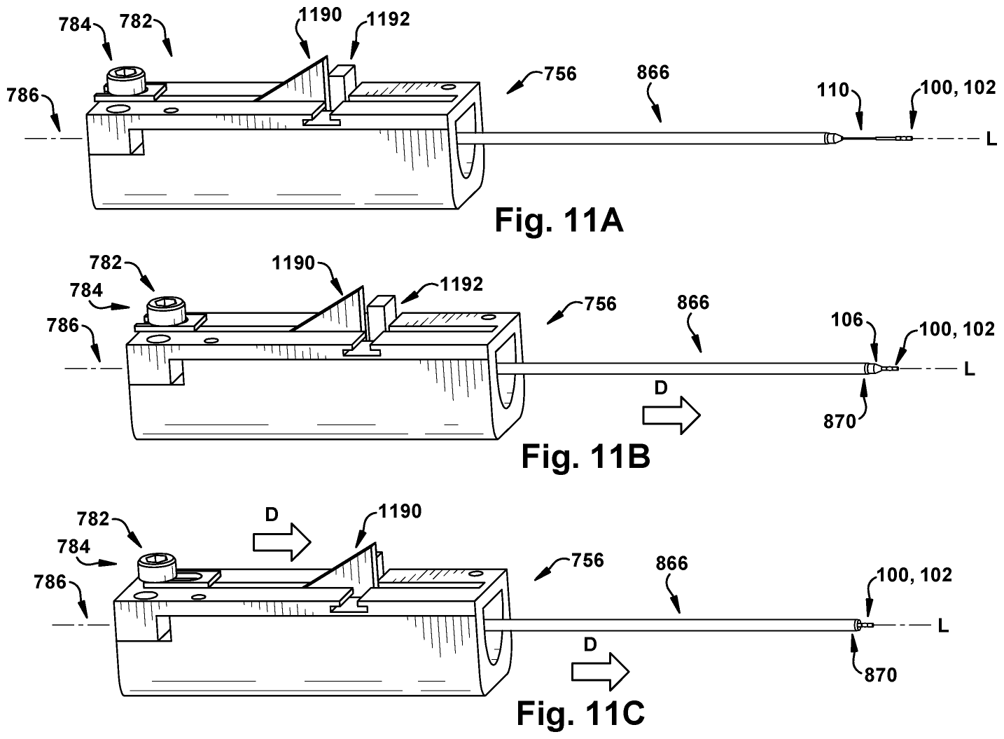

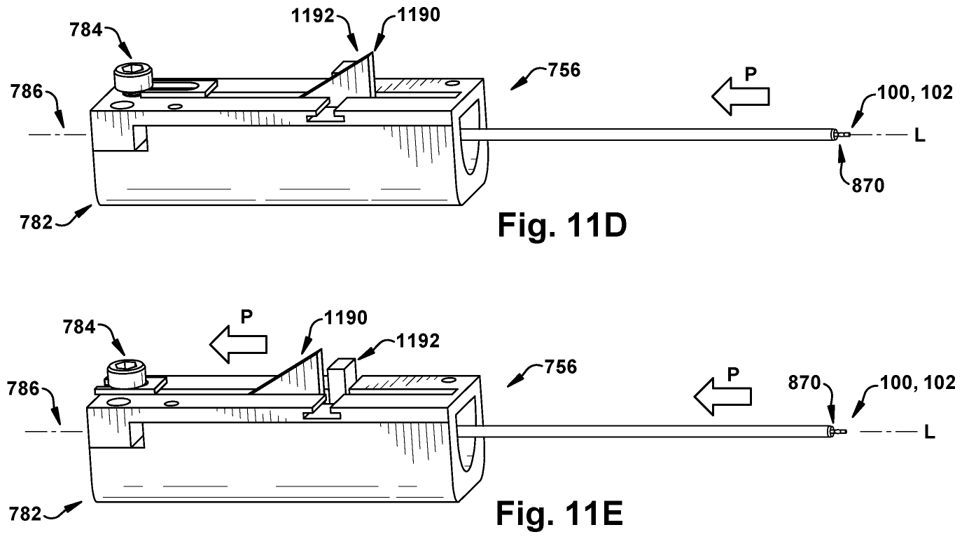

Now that some description has been given of the actions, features, and operation of an aspect of the retrieval apparatus 756 within the body tissue 548 and at the target implantation site 550, reference is made to FIGS. 11A-11E, which schematically depict some example actions, features, and operation of a housing 782 (which can double as a handle for user interaction and may be ergonomically configured accordingly) associated with the retrieval of the medical microdevice 102, anchoring apparatus 100, and/or any desired biopsy sample 1088 as already introduced.

First, as mentioned earlier, the proximal guidewire end 554 is inserted into the stylet lumen 864 to any desired extent. In FIGS. 11A-11E, the proximal guidewire end 554 is inserted all the way through the housing 782 and protrudes from a rear extent thereof. When desired, a clamp 784 of any suitable type (e.g., the turnscrew shown) may be operated to maintain a longitudinal position of the housing 782 with respect to the implanted medical microdevice 102. The stylet lumen 864 is then guided, as represented in FIG. 11B by distal arrow D, toward the target implantation site 550 over the guidewire 110 by passing the stylet lumen 864 distally along the guidewire 110. During this insertion, the nosecone 876, when present, may help with atraumatically penetrating into the body tissue 548. As can be seen in FIG. 11 B, the proximal device end 106 is brought into contact with the distal stylet end 862, and any device notch 880 present may be engaged to ensure, e.g., a rotational orientation of the medical microdevice 102 with respect to the retrieval apparatus 756. Up until the view of FIG. 11B, the housing 782, sheath 866, and stylet 858 have been maintained substantially in an initial longitudinal position with respect to each other.

Then, in FIG. 11C, the distal stylet end 862 is maintained in contact with the proximal device end 106, while the sheath 866 is advanced distally to accept the stylet 858 (with the enclosed guidewire 110), microdevice 102, any biopsy sample 1088 being taken, and any portion of the anchoring apparatus 100 as desired into the sheath lumen 872. This may be done, for example, by advancing a first slider 1190, which is operatively connected to the sheath 866, in the distal direction, as well, as shown by distal arrow D. When the sheath lumen 872 is configured to accept a biopsy sample 1088 therein, the concentric cutting edge 874 may be helpful in coring the body tissue 548 neatly to retrieve a substantially uncrushed/un-torn and relatively undamaged biopsy sample 1088.

It is also contemplated that the sheath 866 could be advanced distally through use of a spring-biased mechanism, a pulley-type pullback slider, direct interaction by the user with the proximal sheath end 868, and/or in any other desired manner. One of ordinary skill in the art will be readily able to provide a housing 782 which provides for the desired mechanical interactions of the components of the retrieval apparatus 756.

Any number of additional sliders or other user-manipulable components may be provided to facilitate retrieval of the medical microdevice 102 and associated anchoring apparatus 100 substantially as shown and described herein. For example, it is contemplated that a reference block 1192 could be provided to help apprise the user of the longitudinal position of the sheath 866, to provide the user with something to rest or brace a finger against during operation of the first slider 1190, or for any other reason. Likewise, it is contemplated that a rotational mechanism (not shown) could be provided to allow a user to impart, e.g. rotational movement centered around the longitudinal axis L, to the stylet 858 in order to, for example, bring the device notch 880 into desired engagement with the proximal device end 106.

As a result of the distal motion of the sheath 866, the retrieval apparatus 756 attains the configuration shown in FIG. 11D, with the microdevice 102 at least partially enclosed in the sheath lumen 872 within the body tissue 548, and the microdevice 102 is laterally surrounded within the sheath lumen 872. Distal movement of the sheath lumen 872 past the anchoring apparatus 100 may be prevented, in some use environments, by at least one of contacting the anchoring apparatus 100 with the distal sheath end 870, accepting at least a portion of the anchoring apparatus 100 into the sheath lumen 872, a visible measurement indication to guide a user's advancement of the sheath 866, and/or a spring-biased or other mechanism which limits distal movement of the sheath lumen 872. The microdevice 102, stylet 858, and any associated biopsy sample 1088 are then maintained within the sheath lumen 872.

As depicted via proximal arrow P in FIG. 11D, the sheath lumen 872, with the microdevice 102 and stylet 858 carried therein, can be withdrawn proximally from the target implantation site 550. As a result, the microdevice 102, any associated biopsy sample 1088, and the stylet 858 are removed, by being carried within the sheath lumen 872, from the body tissue 548. During such retraction, as depicted in FIG. 11D, the stylet 858, sheath 866, and housing 782 remain in substantially fixed longitudinal position relative to one another.

Finally, the action depicted schematically in FIG. 11E may be carried out in the ambient space entirely outside the body tissue 548 of the patient, once the retrieval apparatus 756 which at least partially encapsulates the guide wire 110, medical microdevice 102, and anchoring apparatus 100 there within, has been removed from the patient's body. As represented by proximal arrows P in FIG. 11E, the sheath 866 may be retracted in a proximal direction (e.g., by manipulation of the first slider 1190, or in any other desired manner) a longitudinal distance sufficient to expose the biopsy sample 1088 (when present), microdevice 102, and anchoring apparatus 100 to the ambient space. These components removed from the patient's body tissue 548 may be completely disengaged from the retrieval apparatus 756 (e.g., by release of the guide wire 110 from the clamp 784) and passed to another procedure for testing, analysis, recycling, review, disposal, and/or any other purpose.

Through use of the retrieval apparatus 756, therefore, a medical microdevice 102 and associated structures can be quickly and easily removed from a patient's body, even in a bedside setting. As a result, microdevice 102 retrieval can be made relatively simple and low-cost, as well as comfortable for the patient.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

We claim:

1. A retrieval apparatus for selectively retrieving a medical microdevice and associated anchoring apparatus from a target implantation site of a body tissue, the microdevice including a body having longitudinally spaced proximal and distal device ends and a guidewire extending proximally from the proximal device end, and the anchoring apparatus including an anchor having longitudinally spaced proximal and distal anchor ends, a selected one of the proximal and distal anchor ends being connected to a respective one of the distal and proximal device ends, the anchoring apparatus being selectively expandable between a collapsed insertion condition and an expanded anchoring condition within the body tissue, the retrieval apparatus comprising:
a stylet having proximal and distal stylet ends longitudinally separated by a stylet lumen configured to selectively accept the guidewire; and
a retrieval sheath having proximal and distal sheath ends longitudinally separated by a sheath lumen, the sheath lumen being configured to selectively and concurrently laterally surround the microdevice and the stylet therewithin for removal from the body tissue.

2. The retrieval apparatus of claim 1, wherein the distal stylet end includes a device notch for selectively longitudinally accepting a portion of the proximal device end in a predetermined orientation.

3. The retrieval apparatus of claim 1, wherein at least one of the distal stylet end and the distal sheath end includes a tapered nosecone for atraumatically engaging the body tissue.

4. The retrieval apparatus of claim 1, wherein the distal sheath end includes a concentric cutting edge for coring through the body tissue.

5. The retrieval apparatus of claim 1, wherein the stylet and sheath are mutually coaxially concentric.

6. The retrieval apparatus of claim 1, including a housing operatively connected to the proximal stylet end and the proximal sheath end, the housing being configured for selective manipulation by a user to independently longitudinally move the stylet and the sheath.

7. The retrieval apparatus of claim 6, wherein the housing includes a clamp for grasping at least a portion of guidewire to maintain the device body in relative longitudinal spacing from the housing.

8. The retrieval apparatus of claim 1, wherein the microdevice is implanted in a body tissue with the guidewire protruding proximally through a skin surface from the body tissue into ambient space adjacent the skin surface and is maintained in the implanted position via resistance of the anchoring apparatus, in the expanded anchoring condition, to longitudinal motion of the microdevice, and wherein the stylet is configured for initial engagement with the guidewire in the ambient space and guidance by the guidewire toward the body tissue immediately adjacent the proximal device end.

9. The retrieval apparatus of claim 1, being configured to retrieve a biopsy sample portion of the body tissue immediately adjacent the microdevice by at least partially laterally surrounding the biopsy sample within the sheath lumen concurrently with the sheath lumen laterally surrounding the microdevice and stylet.

10. The retrieval apparatus of claim 1, wherein the sheath lumen is smaller than the anchoring apparatus, when in the expanded anchoring condition.

11. The retrieval apparatus of claim 1, wherein the anchoring apparatus is at least partially surrounded within the sheath lumen concurrently with the sheath lumen laterally surrounding the microdevice and stylet.

12. A method for selectively retrieving a medical microdevice and associated anchoring apparatus from a target implantation site of a body tissue, the microdevice including a body having longitudinally spaced proximal and distal device ends and a guidewire extending proximally from the proximal device end, and the anchoring apparatus including an anchor having longitudinally spaced proximal and distal anchor ends, a selected one of the proximal and distal anchor ends being connected to a respective one of the distal and proximal device ends, the anchoring apparatus being selectively expandable between a collapsed insertion condition and an expanded anchoring condition within the body tissue, the method comprising:
providing a retrieval apparatus including a stylet having proximal and distal stylet ends longitudinally separated by a stylet lumen configured to selectively accept the guidewire, the stylet lumen being configured not to accept the proximal device end thereinto, and a retrieval sheath having proximal and distal sheath ends longitudinally separated by a sheath lumen;
inserting a proximal end of the guidewire into the stylet lumen;
guiding the stylet lumen toward the target implantation site over the guidewire by passing the stylet lumen distally along the guidewire;
contacting the proximal device end with the distal stylet end;
maintaining the distal stylet end in contact with the proximal device end while accepting the stylet and enclosed guidewire into the sheath lumen;
passing the sheath lumen distally along the stylet and into a position within the body tissue such that the microdevice is at least partially enclosed in the sheath lumen within the body tissue;
laterally surrounding the microdevice within the sheath lumen;
preventing distal movement of the sheath lumen past the anchoring apparatus by at least one of contacting the anchoring apparatus with the distal sheath end and accepting at least a portion of the anchoring apparatus into the sheath lumen;
maintaining the microdevice and stylet in the sheath lumen;
retracting the sheath lumen, with the microdevice and stylet carried therein, proximally from the target implantation site; and
removing the microdevice and stylet within the sheath lumen from the body tissue.

13. The method of claim 12, wherein laterally surrounding the microdevice within the sheath lumen includes laterally surrounding the microdevice and at least a biopsy sample portion of the body tissue immediately adjacent the microdevice within the sheath lumen; and wherein the biopsy sample portion is maintained in the sheath lumen, retracted proximally from the target implantation site, and removed from the body tissue concurrently with the microdevice and stylet.

14. The method of claim 12, including:
providing the distal stylet end with a device notch; and
selectively longitudinally accepting a portion of the proximal device end in the device notch in a predetermined orientation when the stylet is in contact with the microdevice.

15. The method of claim 12, including atraumatically engaging the body tissue with a tapered nosecone of at least one of the distal stylet end and the distal sheath end.

16. The method of claim 12, wherein passing the sheath lumen distally along the stylet includes coring through the body tissue around the stylet with a concentric cutting edge of the distal sheath end.

17. The method of claim 12, including
providing a housing for handheld operation by a user;
operatively connecting the housing to the proximal stylet end and the proximal sheath end; and
the user selectively manipulating the housing to independently longitudinally move the stylet and the sheath.

18. The method of claim 17, including grasping at least a portion of guidewire with a clamp of the housing to maintain the device body in relative longitudinal spacing from the housing.

19. The method of claim 12, wherein the microdevice is implanted in a body tissue with the guidewire protruding proximally through a skin surface from the body tissue into ambient space adjacent the skin surface and is maintained in the implanted position via resistance of the anchoring apparatus, in the expanded anchoring condition, to longitudinal motion of the microdevice, and wherein guiding the stylet lumen toward the target implantation site over the guidewire by passing the stylet lumen distally along the guidewire includes:
initially engaging the stylet with the guidewire in the ambient space; and
with the guidewire, guiding the stylet toward the body tissue immediately adjacent the proximal device end.

20. A retrieval apparatus for selectively retrieving a medical microdevice and associated anchoring apparatus from a target implantation site of a body tissue, the microdevice including a body having longitudinally spaced proximal and distal device ends and a guidewire extending proximally from the proximal device end, and the anchoring apparatus including an anchor having longitudinally spaced proximal and distal anchor ends, a selected one of the proximal and distal anchor ends being connected to a respective one of the distal and proximal device ends, the anchoring apparatus being selectively expandable between a collapsed insertion condition and an expanded anchoring condition within the body tissue, the retrieval apparatus comprising:
a stylet having proximal and distal stylet ends longitudinally separated by a stylet lumen configured to selectively accept the guidewire, the stylet lumen being configured not to accept the proximal device end thereinto;
a retrieval sheath having proximal and distal sheath ends longitudinally separated by a sheath lumen, the distal sheath end being configured for selective contact with the anchoring apparatus, the distal sheath end including a concentric cutting edge for coring the body tissue, and the sheath lumen being configured to selectively and concurrently laterally surround the microdevice, the stylet, and a biopsy sample portion of the body tissue immediately adjacent the microdevice therewithin for removal from the body tissue; and
a housing operatively connected to the proximal stylet end and the proximal sheath end, the housing being configured for selective manipulation by a user to independently longitudinally move the stylet and the sheath.
